(19) United States
(12) Patent Application Publication
(10) Pub. No.: US 2026/0021036 A1
(43) Pub. Date: Jan. 22, 2026

(54) FLEXIBLE BIODEGRADABLE MICRONEEDLES

(71) Applicants: SHEBA IMPACT LTD., Ramat-Gan (IL); AZRIELI COLLEGE OF ENGINEERING JERUSALEM, Jerusalem (IL); HADASIT MEDICAL RESEARCH SERVICES AND DEVELOPMENT LTD., Jerusalem (IL)

(72) Inventors: Aiman ABU AMMAR, Baqa Algharbiya (IL); Arsalan ABU MUCH, Baqa Algharbiya (IL)

(21) Appl. No.: 18/854,908

(22) PCT Filed: Apr. 4, 2023
(86) PCT No.: PCT/IL2023/050359

§ 371 Date: Oct. 7, 2024

Related U.S. Application Data
(60) Provisional application No. 63/328,302, filed on Apr. 7, 2022.

(51) Int. Cl. A61K 9/00; A61K 31/635; A61K 47/34; A61M 37/00

(52) U.S. Cl. CPC A61K 9/0021; A61K 31/635; A61K 47/34; A61M 37/0015; A61M 2037/0023; A61M 2037/0053

(57) ABSTRACT

Disclosed herein are flexible microneedle arrays with improved characteristics, such as durability and high drug loading. Further disclosed methods of manufacturing the microneedle arrays, and uses thereof in treatment of a disease or a condition in a patient in need thereof.

### FIELD OF THE INVENTION

The present disclosure relates to flexible microneedle arrays suitable for transdermal delivery of pharmaceutically active agents. More specifically, the present disclosure relates to biodegradable microneedle arrays, comprising a biodegradable polymer and a pharmaceutically active agent, homogenously dispersed therein. The present disclosure also refers to methods of manufacturing of such arrays, as well as their use in treatment of diseases or disorders responsive to said pharmaceutically active agent.

### BACKGROUND

Transdermal drug delivery systems are a useful minimally invasive alternative to other drug administration routes, particularly in the case of poorly water-soluble drugs. Biodegradable polymeric microneedles may be used in controlled-release drug delivery due to their tunable properties and ease of patient self-administration. These systems are a useful minimally invasive alternative to other drug administration routes such as intramuscular (i.m.) and intravenous (i.v.) administration. Such delivery systems are known for their potential to increase the bioavailability of poorly permeable drugs, in particular of biopharmaceutical classification system (BCS) class IV drugs, which are both poorly water soluble and low permeable. Microneedles have been manufactured from a variety of materials, including biodegradable polymers, e.g., polyesters. Polyesters are biodegradable polymers that are often used for sustained drug release because of their intrinsic properties including biocompatibility, biodegradability, and favorable mechanical performance. One particular representative is lactic-co-glycolic acid (PLGA) copolymer, which is widely used in controlled drug delivery. Microneedle arrays for intradermal delivery of PLGA-based microneedle tips has been described in He, M. et J Pharm al. 2020, Sci, 109 (6), 1958-1966. https://doi.org/10.1016/j.xphs.2020.02.009. The publication discloses the manufacturing and biopharmaceutical properties of controlled-release etonogestrel-loaded PLGA microtips, which remain implanted in the skin after application. The optimized drug loading was between 140-166 micrograms, which was apparently sufficient for the high-potency low-dose drug, such as sex hormones.

Therefore, there remains a need in the art to provide microneedles arrays with higher drug loading capacity, e.g., to enable treatment with other drugs, not just sex hormones.

One of the conditions for which limited treatment options are available is congestive heart failure. It imposes a significant medical and economic burden which characterized by high 5-year mortality, reaching a 53% from the moment of diagnosis. From a clinical perspective, fluid overload (e.g., pulmonary congestion, ascites, and lower limp edema) is among the hallmarks of acute exacerbations symptoms, necessitating a frequent patients' hospitalizations for diuretic therapy, loop diuretics (e.g., furosemide) in particular. However, intravenous furosemide is twice as potent as orally administered, due to higher drug bioavailability. On the other hand, i.v. administration necessitating inpatient care, monitoring, and i.v. catheter placement, prone to infections, makes the strategy particularly disadvantageous. Subcutaneous administration, including s.c. pumps, allows more patient mobility, but is still prone to infections and requires close monitoring.

Therefore, there is a particular need in the art to provide an alternative means for administration of loop diuretics, among other drugs suffering from same drawbacks. As demonstrated in the appended examples, microneedle arrays comprising furosemide have been successfully prepared. It has now been unexpectedly found that the microneedle arrays can be prepared with very flexible mechanical properties, to facilitate safe application and wearing by the patient, and yet can be made to contain significantly higher drug loading than previously known in the art, and release the active agent, e.g., furosemide, in sustained fashion over at least 24 hours.

### SUMMARY OF THE INVENTION

Therefore, in one aspect provided herein a flexible microneedle array having exceptional mechanical properties and an increased drug loading capacity, by comprising the drug in the whole of microneedle body. Thus, provided herein a flexible microneedle array comprising a plurality of microneedles and a backing layer, wherein said microneedles comprise a polyester selected from the group consisting cof polylactic acid, polyglycolic acid, poly(lactic-co-glycolic) acid, polycaprolactone, and combinations of any of these, wherein said microneedles further comprise a pharmaceutically active agent that is soluble in a solvent capable of dissolving said polyester, and that is poorly soluble in distilled water at 25° C., and wherein said backing layer comprises an alginate and a polyol. Preferably, the flexible microneedle array is wherein said pharmaceutically active agent is essentially homogenously distributed within said microneedles, such that the concentration of said pharmaceutically active agent at the distal ⅓ portion of said microneedles is between 50% and 150% of that in the proximal ⅓ portion of said microneedles, relative to the base of said microneedles and said backing. In some embodiments, the flexible microneedle array is wherein said pharmaceutically active agent and said polyester are individually or concomitantly essentially homogenously distributed within said microneedles, such that the concentration of said pharmaceutically active agent and/or said polyester at the distal ⅓ portion of said microneedles is between 50% and 150% of that in the proximal ⅓ portion of said microneedles, relative to the base of said microneedles and said backing layer. In some further embodiments the flexible microneedle array is wherein said backing layer is flexible such that no cracks are formed when said microneedle array is bent over a central axis of said microneedle array by repetitively bringing together the opposing edges of the backing layer over 50 times. Preferably, the flexible microneedle array is wherein said solvent capable of dissolving said polyester comprises chloroform, dichloromethane, or acetone. In some preferred embodiments, the flexible microneedle array is wherein a weight ratio between said polymer and said pharmaceutically active agent is between 1.5:1 and 5:1. In some further embodiments, the flexible microneedle array is wherein a weight ratio between said polymer and said pharmaceutically active agent is between 2:1 and 4:1. The flexible microneedle array may be wherein pharmaceutically active agent is selected from the group consisting of furosemide, levosimendan, dobutamine, amiodarone, metoprolol, lidocaine, and chlorhexidine. Currently preferably, the flexible microneedle array is wherein pharmaceutically active agent is furosemide. In some embodiments, the flexible microneedle array is wherein a weight ratio between said alginate and said polyol in said backing layer is between 1:1 and 3:1. Preferably, the flexible microneedle array is wherein a weight ratio between said alginate and said polyol in said backing layer is between 1.8:1 and 2.3:1. In some further embodiments, the flexible microneedle array is wherein said polyol is selected from the group consisting of glycerin, sorbitol, xylitol, and combinations of any of the above. Preferably, the flexible microneedle array is wherein said polyol is glycerin. In some embodiments, the flexible microneedle array is wherein said polyester is poly-lactic-co-glycolic acid, wherein said pharmaceutically active agent is furosemide, and wherein said polyol is glycerin. Preferably, the flexible microneedle array is wherein a weight ratio between said poly-lactic-co-glycolic acid and said furosemide is between 2:1 and 4:1. Further preferably, the flexible microneedle array is wherein a weight ratio between said alginate and said glycerin in said backing layer is between 1.8:1 and 2.3:1. In a further aspect provided herein is a method of manufacturing a microneedle array, comprising combining on a microneedle array mold an organic solution of a polymer forming the microneedles and an aqueous solution of a polymer forming a backing layer of said microneedle array. Preferably, the method comprises providing a mold for microneedle array, providing a microneedle precursor solution comprising a mutual solution of a polymer and a pharmaceutically active agent in a solvent, applying said microneedle precursor solution to said mold, optionally degassing and/or partially evaporating said microneedle precursor solution on said microneedle mold, and applying an aqueous backing layer solution, wherein said aqueous backing layer solution comprises an alginate and a polyol, and wherein said applying of said aqueous backing layer solution effects forming microneedles from said microneedle precursor solution in said mold, said microneedles comprising said polymer and said pharmaceutically active agent. In some embodiments, the method further comprises drying said mold after said applying of said aqueous backing layer solution. In further embodiments, the method further comprises sterilizing said microneedle array. In some further embodiments, the method further comprises packing said microneedle array in a suitable package. In a further aspect provided herein a microneedle array as defined herein, for use in treatment of a heart disease or a vascular disease in a patient in need thereof. Preferably, the microneedle array is wherein said microneedle array is applied to the skin of said patient. Further preferably, the microneedle array is wherein said microneedle array is applied to the patient in need thereof in a frequency selected from the group of thrice daily, twice daily, once daily, thrice weekly, twice weekly, and once weekly.

### BRIEF DESCRIPTION OF FIGURES

FIG. 1 presents a micrograph of a microneedle array according to an embodiment of the invention.

FIGS. 2A to 2D demonstrate scanning electron micrographs of microneedles arrays of the several tested formulations.

FIG. 3 demonstrates a photograph of a microneedle array bent over its central diagonal axis.

FIG. 4 demonstrates the size of the pores formed in layers of substrate perforated with a microneedle array according to an embodiment of the invention.

FIG. 5 demonstrates a differential scanning calorimetry thermogram of several components of the microneedle array and an array according to an embodiment of the invention.

FIG. 6 depicts the powder X-ray diffraction (PXRD) patterns of furosemide alone and loaded into a microneedle array according to an embodiment of the invention.

FIG. 7 presents an attenuated total reflectance Fourier-transformed infra-red (ATR-FTIR) spectra of PLGA, furosemide, and an array according to an embodiment of the invention.

FIG. 8 presents a chart of in vitro drug release from an array according to an embodiment of the invention. Inset shows the drug release profile over the initial 6 h.

### DETAILED DESCRIPTION

Thus, in a first aspect, provided herein a microneedle array for the delivery of a pharmaceutically active agent into and/or under the skin of a patient in need thereof. The “microneedle array” is interchangeably referred to herein as just “array”, “microarray”, “microneedles”, and in like terms, unless the context clearly dictates otherwise. The microneedle array, as described in greater detail below, comprises a plurality of micro-sized needles, disposed on a backing layer. The microneedle array is usually very flexible, due to its structure as elaborated below, such that it allows unhindered handling of the array, easy application to almost any part of body, e.g., of varying curvature, and may thus provide an improved comfort to the patient, which in turn can lead to improved patient compliance. As demonstrated in the appended examples, a microneedle array prepared according to one embodiment of the invention could be readily bent without impairing its physical integrity, for over 300 times. Thus, provided herein is a microneedle array that is flexible, e.g., such that it may be elastically bent along its central axis, by bringing together the opposing edges of the backing layer, multiple times, e.g., over 50 times, or over 100 times, or over 150 times, or over 200 times, or over 250 times, or even over 300 times, while remaining essentially intact. The microarray that remains essentially intact usually contains the identical number of the microneedles after the multiple bending challenge as the original array has, and preferably it contains no cracks, splits, or other imperfections or defects in the backing layer. However, the microarray may fail after bending as described, e.g., over 50, 100, 150, 200, 250, 300, or more than 300 times. Currently preferably, the microneedle array does not fail after bending as described, for 300 times,

The microneedle array comprises the microneedles and a backing layer supporting the microneedles. Due to the choice of materials that make up the microarray, as described in greater detail below, it is possible to retain sufficient mechanical strength to enable the microneedles to puncture the corneal layer of the skin (stratum corneum), and on the other hand, to enable this extraordinary flexibility; all these advantages being present without compromising the loading capacity of the array as drug delivery system.

The microneedles are usually evenly distributed throughout the array, e.g., they are essentially evenly spaced one from another, although arrays with irregularly distributed microneedles may also be envisaged. The even distribution may be expressed in a value called array pitch, or just “pitch”, which, as used herein, should be construed as an average distance between microneedle tips. The pitch may usually be a measure of microneedles density in the array. It is evident that depending on the base dimensions, the pitch would vary accordingly, with the densest possible configuration being when the pitch is equal to the base dimension of the microneedle. The pitch may also be adjusted to afford the flexibility of the microneedles array, such that to avoid friction between the array elements upon bending or other handling. For example, when microneedles' base is about 200 micrometers, the pitch may preferably vary between 300 and 700 micrometers, further preferably between 400 and 600 micrometers. Without being bound by a particular theory it is believed that the denser microarrays would allow higher absolute drug loading per a unit of area, due to a larger number of microneedles. As used herein the term “absolute drug loading”, used herein interchangeably with the terms “loading capacity” and the like, should be construed as amount of drug in weight units, e.g., in micrograms or in milligrams, per unit or area, or per one array, unless the context clearly dictates otherwise, e.g., when discussing the relative drug loading in a formulation, which is usually expressed in percentage.

The microneedles of the array, in their turn, may be of any suitable shape and size to perform their basic functions, which include puncturing the corneal layer of the skin and substantially penetrating beneath it. Thus, the height of the microneedles is selected such that it would be greater than, e.g., 100 micrometers, to ensure penetration of the corneal layer of the skin. On the other hand, it may be advantageous to provide microneedles of greater dimensions, of greater volume in particular, as this would allow increasing absolute drug loading. However, large microneedles may cause significant distress to the skin, inter alia by reaching the enervated tissues and triggering nociception. Such upper limit dimensions are readily known in the art. Depending on the base size, the microneedles may have the height of between 300 and 1000 microns, preferably between 400 and 600 microns. The base of the microneedles may be of any suitable shape, e.g., a circle, an oval, an ellipse, a polygon, or an irregular figure. However, to maximize the force transduction when it is applied onto the microneedles' array, it is preferable that the base be a regular geometric figure, e.g., a circle, or an equilateral polygon, such as an equilateral triangle, a square, a hexagon, etc. The microneedles may thus be in a form of pyramids or cones. Preferably, the microneedles have the tip projection positioned at the geometrical center of the base, e.g., at diagonals crossing point for the square base, or the center of the circle. The microneedles may also be of a more complex shape, e.g., prism or cylinder at a base, and a pyramid or a cone at the tip, provided that the tip is sufficiently thin to enable puncturing the skin upon application of pressure onto the microarray. Embodiments wherein at least part of microneedles have different shape from the others are also envisaged. In some currently preferred embodiments, the microneedles are in form of pyramids.

The microneedles' array may be of any suitable size and shape per se. Generally, the number of microneedles per array and their distribution density may be such that the microarray has comfortable dimensions for application and wearing, and contains sufficient number of microneedles to carry and administer the desired dose of the drug. For example, the microneedles array that is demonstrated in the examples below, has 100 microneedles evenly spaced on a 5 mm per 5 mm square-shaped area. The number of microneedles per array, may therefore be, depending on the drug loading, from 100 microneedles as exemplified herein, and up to 200,000 microneedles, for a microneedle array of exemplary dimensions of about 20 cm×20 cm and slightly increased microneedles' density. Larger arrays are also envisaged for applications requiring larger doses, or longer duration of release.

The microneedles comprise an active pharmaceutical ingredient (API), also referred to herein interchangeably as “pharmaceutically active agent”, and also in terms such as “drug”, “active”, and like. The microneedles also comprise a polymer. The polymer is an important part of the microneedles, as it serves as the carrier for the drug and controls its release upon penetrating into the skin, and on the other hand, it should have the mechanical properties suitable to puncture the skin, and to exhibit little or no deformation upon application. A microneedle comprising the polymer should therefore have sufficient mechanical strength to withstand the force of no less than 2 N, applied coaxially with the microneedles, e.g., onto the backing layer, with a strain of no more than 50%. Particularly, and currently preferably, the mechanical properties should allow the microneedles to withstand the applied force of no less than 10 N with a strain of no more than 30%.

Given the nature of the microneedle array, the polymer is a biodegradable polymer. Usually the polymer is a polyester, a polyanhydride, or a polyester-anhydride. Other biodegradable polymers may include polyurethanes, polyphosphoesters, polyurethanes, and polyamides (e.g., polyamino acids). Preferably the polymer is a polyester and comprises residues of lactic and/or glycolic acid; these polymers are generally known under common name “polylactides” or “polyglycolides”. The polymer may thus be preferably selected from polylactic acid, polyglycolic acid, and poly-lactic-co-glycolic acid. Further polyester polymers comprise further polyhydroxyalkanoates, such as polyhydroxybutyrate, polyhydroxyvalerate, polycaprolactone, and others, such as polydioxanones. A co-polymer comprising repeating units of any of the enumerated polyesters, as well as derivatives thereof, may also be used, provided that it is soluble in at least one solvent that dissolves also the pharmaceutically active agent, and that it is insoluble or poorly soluble in water. When the polymer is a copolymer, e.g., a poly-lactic-co-glycolic acid, it may contain any suitable ratio of monomers, as known in the art and technically feasible. A blend of polymers as described herein, may also be used. In currently preferred embodiments, the microneedles comprise a polymer which is a polyester, selected from the group consisting of polylactic acid, polyglycolic acid, poly(lactic-co-glycolic) acid, polycaprolactone, and combinations of any of these. In some embodiments, the microneedles consist essentially of the API and the polymer. The terms relating to solubility, e.g., “poorly soluble”, “freely soluble”, and like, should be construed according to the meaning and definitions as known in the art, e.g., as defined in Unites States Pharmacopeia, e.g., USP-32, or the International Pharmacopeia, 10th edition. The term “poorly soluble” should be construed as having a solubility below the “sparingly soluble” definition. A reference table of definitions is presented in the table below:

| Term | Soluble in |
|---|---|
| Very soluble | Less than 1 part |
| Freely soluble | From 1 to 10 parts |
| Soluble | From 10 to 30 parts |
| Sparingly soluble | From 30 to 100 parts |
| Slightly soluble | From 100 to 1,000 parts |
| Very slightly soluble | From 1,000 to 10,000 parts |
| Practically insoluble | More than 10,000 parts |

The solubility definition in the table above, unless otherwise indicated, relates to the approximate solubility of a substance that is evaluated at 20° C. The expression “part” in the table describes the number of millilitres (mL) of solvent represented by the stated number of parts in which 1 gram (g) of solid is soluble.

The polymer in the microneedles layer may have any suitable molecular weight and polydispersity, provided, that it forms a readily pourable solution in the solvent as defined herein, e.g., at a concentration between 0.5% and 10% by weight, e.g., between 0.5%, or 1%, or 1.5%, or 2%, or 2.5%, or 3%, or 3.5%, or 4%, or 4.5%, or 5%, and 20%, or 15%, or 12%, or 10%, or 9%, or 8%, or7%, or 6.5%, or 6%, or 5.5%, by weight. The polymer is thus selected such that a viscosity of an acetone solution (or, alternatively, a solution in other solvent as enumerated below) at a concentration by weight as defined herein, is between 5 and 5,000 mPa*s, such that the viscosity is such, that the solution flows freely into and fills the selected microneedles mold or the selected geometry, as described herein. Preferably, the polydispersity of the polymer is less than 4, further preferably less than 3. The molecular weight of the polymer may usually be between 4,000 and 200,000 Dalton.

The microneedles thus further comprise a drug. The drug may be usually selected based on several important properties, including, but not limited to, the elimination half-life, the therapeutic dose, the desired concentrations' range of the drug in the body, etc. As to the pharmacokinetic parameters, the drug should preferably have a short elimination half-life, e.g., between 10 minutes and 5 hours. The therapeutic dose should be accommodated in the microneedle array, and readily controlled by the array's dimensions and density, as described above.

As described in further detail below, the drug should be selected such that it is soluble in at least one solvent that dissolves appreciable amounts of the polymer used in the microneedles. Exemplary solvents that dissolve both the drug and the polymer include chloroform, dichloromethane, acetone, and any organic solvent comprising a mixture of one or more of the above with each other or with other solvents. Without being bound by a particular theory it is believed that this property of solubility in a mutual solvent would allow an essentially homogenous distribution of the drug inside the microneedle, regardless of the final solid state wherein the drug is present in the microneedle, i.e., a dispersion of amorphous powder, a dispersion of a crystalline powder, a solid solution in the polymer, and any combination of these states, i.e., some of the drug being present in a crystalline form, some as amorphous powder, and some amount of the drug being dissolved in the polymer. The drug should preferably also have a comparable solubility in other solvents to the polymer used, such that the drug and the polymer remain in a solution together, and if the solvent conditions are changed and the polymer is precipitated, the drug would precipitate as well at very similar conditions. Therefore, the drug should have a limited solubility in distilled water, e.g., at room temperature (between 15° C. and 30° C.). The drug may have in improved solubility in aqueous buffer solutions, particularly in buffers of physiological pH values, e.g., between 5.0 and 7.5, than its solubility in distilled water at same temperature. The drug should therefore be soluble in distilled water at amounts of less than 1 mg per liter, preferably less than 0.5 mg per liter, and further preferably less than 0.1 mg per liter. This property of similar solubility in at least a pair of solvents is advantageously utilized in the manufacturing of the microneedle arrays, as described in greater detail below, and it is currently believed, without being bound by it, that the property affords manufacturing of the microneedles with uniform distribution of the drug in the microneedles, as well as to limit the presence of the drug in the base of the microneedle array, e.g., in the backing layer. Other characteristics of the drug may also include the log P of between 1.5 and 7. Some specific drugs suitable for the use in the microneedle arrays include diuretics, e.g., furosemide, and also levosimendan, dobutamine, amiodarone, metoprolol, lidocaine, and chlorhexidine. Other compounds include steroids, hormones, peptides and proteins, including antibodies. Further pharmaceutically active agents may be readily incorporated in the microneedles' array, as described herein, regardless of their pharmacological properties, provided they conform to the pharmaceutical requirements herein. Currently preferred drug for the microneedle arrays is a loop diuretic, e.g., furosemide.

The drug may, therefore, be usually essentially homogenously distributed within the microneedles. This homogenous distribution may be reflected in that that the concentration of the drug at the tips of the microneedles is similar to, e.g., roughly the same as or in the range of between 50 and 150% of that, in the proximity to the base of the microneedles. The essentially homogenous distribution of the drug within the microneedles may advantageously be utilized to provide a controlled release of the drug from the microneedles, considering the biodegradable nature of the polymer. This equal “filling” of the microneedles allows utilizing larger volumes for drug loading, increasing the deliverable dose. Additionally, it is currently believed, without being bound by a particular theory, that microneedles with essentially homogenously distributed active agent possess more uniform mechanical properties, thus facilitating the application of the microneedle array, and to do so in a more reproducible and uniform way, i.e., the number of microneedles in the array penetrating the skin can be maximized. Thus, preferably, both the drug and the polymer are essentially homogenously distributed within the microneedles, that is, both are roughly at the tips of microneedles at concentrations between 50 and 150% of the proximity to the base of the microneedles. In this connection, the terms “tip” portion or the “tips” of the microneedles, and the like, may be taken as the portion of about ⅓ of the microneedle length distal to the backing layer, and the “base” portion or the “proximity to the base” and the like, as the portion of about ⅓ of the microneedle length proximal to the backing layer, both in reference to the backing layer of the microneedle array.

The weight ratio between the polymer and the drug in the microneedles is usually between 1.5:1 and 5:1, preferably between 2:1 and 4:1, by weight. This weight ratio in favor of the polymer may ensure the adequate formation of matrix suitable both for the controlled release and having suitable mechanical properties to puncture the skin, e.g., to withstand an apical pressure of 20 N without breaking. The absolute amounts of the drug and the polymer will be dictated by the absolute drug loading, by the dimensions of the array and of the microneedles, and the processing parameters of the manufacturing. The absolute drug loading will preferably be such that it is sufficient to maintain a desired blood plasma concentration for a predetermined time interval, e.g., for 24 hours or for 48 hours. For example, when the drug is furosemide, the absolute drug loading may vary between 1 and 10 mg/array of 100 microneedles, or between 10 mg and 100 mg per array of 1000 microneedles. Higher-end drug loading values may be used for the drugs that are essentially present in dispersed form in the microneedles, provided that the amount of polymer is sufficient for the controlled release of the drug for the desired time interval.

The drug is releasable from the microneedles upon exposure to an aqueous environment, e.g., an artificial fluid for dissolution testing, or body fluid, if the array is applied to the skin. The drug is preferably controllably releasable from the microneedles, e.g., at a specific rate, and not immediately. The rate of drug release may be controlled by the formulation of the microneedles, and may release a fraction of drug as function of time, the fraction being proportional to various parameters, e.g., to the square root of time, or to the time in any other power between 0.3 and 1. An initial rapid release may also be observed. This initial rapid release may account for between 5% to 40% of the deliverable dose, released between 15 minutes to 2 hours. The average duration of the release may be adjusted according to the needs, but generally the microneedles release at least 80% of the drug in controlled manner, as described herein and demonstrated in the appended examples, e.g., during 24 hours-interval, or during 12 hours-interval, or during 36 hours-interval, or during 48-hours interval, or during 72-hours interval. Depending on the pharmaceutically active agent and its potency, the microneedles may be formulated to release the at least 80% of the drug in controlled manner within one week, two weeks, or even one month. The release duration may be conveniently determined in dissolution studies, when the microneedle array is subjected to a liquid medium at sink conditions (i.e., when the amount of the medium is sufficient to completely dissolve at least 300% of the drug present at the test, preferably at least 1000% of the drug). Whereas these dissolution studies may be indicative of the inherent controlled release potential of the microneedles, the actual drug release rate in vivo may be significantly slower, and the release duration consequently significantly longer. Without being bound by a particular theory, it is believed that the in vivo release may be slower that the in-vitro under sink conditions, due to sub-sink momentary conditions at the application site.

As disclosed above, the microneedle array comprises a backing layer. The backing layer of the microneedle array may usually be made of a polymer, preferably a water-soluble polymer. It has now been unexpectedly found that utilizing sodium alginate as a water-soluble polymer and a polyol in the base layer it is possible to obtain microneedle arrays with excellent flexibility, durability, and tight attachment of the microneedles to the backing layer, despite the differences in polymer nature and separate phases of the polymers of microneedles and the backing layer. Thus, preferably the backing layer comprises an alginate. As used herein, the term “alginate” refers to a polymer having alginic acid backbone, and at least a part of the carboxylic acids thereof being ionized and forming a salt. As known in the art, alginic acid is a polysaccharide linear polymer formed by beta-D-mannuronate and alpha-L-guluronate, via 1-4 glycosidic linkage. Alginic acid may have alternating mannuronate and guluronate blocks, and sometimes may have homopolymeric blocks of polymannuronate and polyguluronate. Alginic acid is available from a variety of sources, e.g., from various species of multicellular algae, or from bacterial sources. It is currently believed that the particular source of alginate is immaterial. Preferably, the salt of the alginic acid is with an alkali metal or an alkali earth metal. Most preferably, the alginate is sodium alginate. Also preferably, alginate is a fully neutralized alginate, e.g., comprising equal amount of equivalent of the metal cation and the carboxylic acid residues. Thus, in particularly preferred embodiments, the alginate is sodium alginate. The alginate in the backing layer may have any suitable molecular weight and polydispersity, provided that it forms a readily pourable solution in water, at a concentration between 0.5% and 10% by weight. The alginate is thus selected such that a viscosity of an aqueous solution at a concentration between 0.5% and 10% by weight is between 5 and 5000 mPa*s. Preferably, the polydispersity of alginate is less than 4, further preferably less than 3. The molecular weight of alginate may usually be between 5000 and 200000 Dalton. One advantage of utilizing alginate in the backing layer, in addition to others enumerated below, is that when wetted by the microwounds, it may facilitate adhesion of the microarray to the skin and its retention in place. Other advantage is that it may also be washed off, after the microneedles are sufficiently implanted, to improve the patient's experience.

In addition to alginate, the backing layer further comprises a polyol. Without being bound by a particular theory it is currently believed that the polyol in the backing layer serves as a plasticizer for the alginate. Additionally, it is believed that polyols may also compatibilize the interface between the polymers, i.e., the alginate and the polymer of the microneedles, particularly if the polymer is a polyester such as polylactic acid, polyglycolic acid, poly(lactic-co-glycolic) acid, polycaprolactone, or combinations thereof. The polyols in the backing layer may be mono- or oligosaccharides, or smaller molecules, such as glycerin, propylene glycol, and like. Some currently preferred polyols include glycerin, sorbitol, and xylitol. The weight ratio between the alginate and the polyol in the backing layer may usually between 1:1 and 3:1, e.g., between 1.8:1 and 2.3:1.

The formulation of the microneedle array as described herein gives rise to flexible arrays. As demonstrated in the appended examples, the microneedle arrays could be handled to extreme limitations, without impairing the integrity of the array. This flexibility is translated into that, that the arrays could be bent over completely and repetitively without emergence of cracks or other defects, up to over 300 times. As described briefly above, the flexibility of the array may usually be such that bending of the array along an axis traversing a central point, i.e., a central axis, including repetitive bending along this axis, effects predominantly elastic deformation and cause no or little harm to the array. The bending axis can be chosen as practically reasonable, e.g., along a diagonal of the array, or by along the line traversing the medians of the array sides and the central point. The array withstands the bending along a central axis of the array, e.g., by bringing together the opposing edges of the backing layer over the central axis, such that the backing layer remains intact, and the microneedles remain in place. It is currently believed, without being bound by a theory, that this extraordinary flexibility and durability of the array at least partially arises from the selected components of the array; it is further believed, without being bound by a theory, that the choice of the microneedle polymer and the drug, both of which can be dissolved in a mutual solvent, creates uniform microneedles, e.g., upon application of the backing layer solution, as described in greater detail below, and an interface is created wherein the microneedle tough polymer is at least partially blended with alginate. This hybrid interface may afford attachment of the microneedles to the backing layer and durability during repetitive stress, whereas the flexibility may be imparted by the properly plasticized backing layer, suitably compatibilized with the microneedles' polymer. Depending on the intended use of the microneedles, it may be possible to affect the nature of the interface, e.g., by relative concentrations of the polymers and some process parameters, such that the backing layer could be washed off completely after application, leaving the microneedles in the skin after the application. Alternatively, the interface may be of sufficient resilience to allow removal of the microneedle array, should the need be, particularly if additional layers are places on the backing layer, as described below.

Further layers may be applied to the microneedle arrays, over the backing layer, e.g., a woven or non-woven tissue layer to prevent accidental adherence of the array from the back side thereof, an aluminum foil, e.g., to prevent residual moisture loss, and others, as known in the art.

Other components may be present in the microneedle arrays as described herein, in addition to ones described above. The microneedles and the backing layer, may comprise, both or individually, any one of the following excipients below. The microneedle arrays may include surface active agents, e.g., to modify the release of the drug from the microneedles, or to facilitate the wash-off of the backing layer, such as polysorbates, sorbitan fatty acid esters, poloxamers, polyoxyethylated fatty acid ethers, and others. The microneedle array may comprise fillers, e.g., to increase the mechanical strength of the microneedles, or to render the backing layer less tacky in dry form. Antioxidants and preservatives may be used, e.g., to slow down the degradation of the active ingredient or the polymers, and to inhibit microbial growth in the final dosage form. Buffers may be used, in particularly in the backing layer, to maintain the pH of the alginate, and to decrease the rate of interphase degradation. Lubricating agents, usually poorly miscible with the other components of the microneedle array, may be used to facilitate detachment of the microarrays from the molds. Suitable excipients are enumerated in various compendia and are well-known to the skilled artisan, e.g., appear in the Handbook of Pharmaceutical Excipients (Rowe, R. C., et al. “Handbook of Pharmaceutical Excipients, 7th edn, 784-790.” (2012).), or in the Internet site of the US Food and Drug Administration, as currently listed in Inactive Ingredients in Approved Drug Products database.

Thus, in a further aspect, provided herein a method of manufacturing of microneedle arrays, comprising a step of exposing a mutual solution of a polymer and a pharmaceutically active agent in an organic solvent, e.g., a microneedle precursor solution, to an aqueous solution of backing layer polymer. This exposure of the organic solution of the microneedle polymer to aqueous environment effects phase transition of the polymer, which is usually already present in a microneedle mold, such that microneedles are formed of the polymer. Therefore, since the pharmaceutically active agent has similar solubility properties as described above, it co-precipitates in the microneedles with the polymer, forming an essentially homogenous microneedle in the mold.

Therefore, the method of manufacturing comprises providing a microneedle array mold. The mold may be produced as known in the art, e.g., as described in WO2015122838, in any suitable material, preferably in a durable flexible material, e.g., in polydimethylsiloxane, polyvinyl siloxane (PVS), or the mold can be 3-D printed. The mold may have receptacle element, with a plurality of cavities at the bottom thereof. These cavities are usually essentially perpendicular to the base of the receptacle and are reciprocal in shape to the microneedles to be formed. The cavities in the receptacle may therefore have the shape and the density as described above for the microneedles. The mold may be of a size of an actual microneedle array, or may be at any suitable larger size, e.g., to enable manufacturing of multiple microneedle arrays concomitantly. In this case the cavities in the mold may be grouped into cavity groups corresponding in size to the contemplated microneedle array. These groups may be separated from one another, e.g., by an increased distance between the cavities, or by a protrusion or series of protrusions encircling the groups of cavities on the bottom of the receptacles.

The mold is usually filled with a microneedle precursor solution. This solution comprises a pharmaceutically active agent and the polymer, in essentially completely dissolved form. Preferably the polymer is completely dissolved and forms a true solution in the microneedle precursor solution. The drug may also be completely dissolved, or may be partially dispersed in form of colloidal particles. Preferably, the amount of the drug dissolved in the microneedle precursor solution is above 50% of the total drug amount by weight, introduced into the solution, further preferably above 60%, or above 70%, or above 80%, or above 90%, or above 95%, or above 99%, or above 99.9%. The solvent used in the microneedles precursor solution is therefore capable of dissolving both the drug and the polymer. There are several solvents capable of dissolving the polymers suitable according to the present invention, including polyesters. Some such solvents comprise chloroform, dichloromethane, and acetone. The solvent may also be a solvent mixture that comprises these solvents, and may further comprise additional solvents that do not significantly alter the solubility of the polymer and the drug in the solvent mixture or in water. To prepare the microneedle precursor solution, the polymer and the drug are added consecutively or concomitantly to the solvent or to the solvent mixture, and mixed using mixing means as known in the art, e.g., using a suitable mixer, such as mechanical overhead mixer equipped with an impeller. The mixture is mixed until complete dissolution. The mixture may be heated to facilitate the dissolution of the polymer and/or the drug. Preferably, the heating is below the temperature whereat the pharmaceutically active agent begins decomposition, and in some cases should be avoided altogether. When the mixture is heated, it may be heated to a temperature between 25° C. and a temperature several degrees lower than the boiling temperature of the solvent or of any of its component. Therefore, when chloroform or acetone are present in the solvent, the mixture may be heated to a temperature between 25° C. and 52-55° C., and when dichloromethane is present in the solvent, the mixture may be heated to a temperature between 25° C. and 36-37° C.

The obtained microneedle precursor solution is applied to the mold. Applying of the solution to the mold may be carried out by transferring the solution onto the mold. The mold is preferably levelled prior to transferring of the microneedle precursor solution thereon, to avoid overflow of the receptacle and to ensure an equal distribution of the solution on the mold. The solution may be fed into the mold as known in the art, e.g., by pouring it onto the mold via one or more outlets, by transferring the solution, e.g., with a suitable pump, onto the mold. The solution may also be fed into the mold via a manifold feeder positioned over or inside the cavities of the mold. The particular means to apply the solution will be dictated by the needs of the process. The amount of the solution may be such that it is sufficient to fill the cavities of the mold; that is, the volume of the microneedle precursor solution applied is usually equal or slightly larger than the combined volume of the microneedles.

The mold with the applied solution thereon may be preferably degassed, e.g., by placing the mold into a vacuum chamber. Degassing of the solution in the mold usually accomplishes several tasks: one is to remove the trapped air bubbles at the tips of the cavities in the mold, and thus ensure uniform formation of the microneedles. Another goal is to remove the air bubbles to ensure uniform coverage of the mold by the microneedle precursor solution. A further goal is to effect a partial evaporation of the solvent, as need may be.

A backing layer solution is also prepared for the application to the mold. To prepare the backing layer solution, the alginate and the polyol are added consecutively or concomitantly to the solvent or to the solvent mixture, and mixed using mixing means as known in the art, e.g., using a suitable mixer, such as mechanical overhead mixer equipped with an impeller. When a liquid is used as the polyol, e.g., glycerin, the components may be added simultaneously, e.g., the alginate powder may be pre-mixed with glycerin to wet the powder and assist in its dispersion in water. The mixture is mixed until complete dissolution. The mixture may be heated to facilitate the dissolution of the components of the backing layer solution. Preferably, the heating is to a temperature between 25° C. and 60° C.

Once the microneedle precursor solution is applied onto the mold, the aqueous backing layer solution is applied consecutively onto the mold. The presence of water in the backing layer solution changes the solvent composition of the microneedle precursor solution, and the polymer and the drug precipitate together into the microneedle cavities. Additionally, an interface (or in fact an interphase) of varying thickness is formed between the microneedle precursor solution and the backing layer solution, wherein the polymers are at least partially blended together, particularly due to the presence of polyols in the backing layer solution. When the solvent of the microneedle precursor solution is miscible with water, e.g., acetone, it is dissolved in the backing layer solution that is being applied onto the mold. When the solvent of the microneedle precursor solution is only partially miscible with water, e.g., chloroform and dichloromethane, they are extracted into the backing layer solution, or forced through it by the forming interface of the microneedles' polymer and the backing layer polymer, i.e., the alginate. These solvents are evaporated later in the process, e.g., during drying of the mold. The mold may be dried by means known in the art, e.g., in a suitable oven. Preferably, that the process be performed on a levelled surface, as mentioned above, and therefore drying may be carried out in a vacuum oven. Additionally, the drying may be performed in a desiccator. Drying may be performed until the composition in the mold is completely solidified and non-tacky. Suitable in-process quality controls may be employed to determine the endpoint of drying of the microneedles array, e.g., gravimetry, or water determination, e.g., via Karl-Fischer titration.

The dried microneedles array may be detached from the mold, and may further be processed as needed, e.g., further dried, sterilized, packaged, etc.

Thus, provided herein a method of manufacturing a microneedle array, comprising providing a mold for microneedle array, providing a microneedle precursor solution comprising a mutual solution of a polymer and a pharmaceutically active agent in a solvent, applying said microneedle precursor solution to said mold, optionally degassing and/or partially evaporating said microneedle precursor solution on said microneedle mold, and applying an aqueous backing layer solution, wherein said aqueous backing layer solution comprises an alginate and a polyol, and wherein said applying of said aqueous backing layer solution effects forming microneedles from said microneedle precursor solution in said mold, said microneedles comprising said polymer and said pharmaceutically active agent. The method may further comprise drying said mold after said applying of said aqueous backing layer solution.

The obtained microneedles array may be used in treatment of patients in need thereof. For example, the microneedles arrays may be used to treat or ameliorate the symptoms of a disease or disorder responsive to the pharmaceutically active agent in the microneedles array. When the drug is furosemide or other loop diuretic, the microneedles array may be used to treat or ameliorate the symptoms of a heart disease or a vascular disease, e.g., congestive heart failure, an abnormal heart rhythm, an aorta disease, e.g. Marfan syndrome, a congenital heart disease, a coronary artery disease, deep vein thrombosis and pulmonary embolism, heart attack, heart failure, a heart muscle disease (cardiomyopathy), a heart valve disease, a pericardial disease, a peripheral vascular disease, a rheumatic heart disease, and stroke.

The microneedle arrays are usually administered to a patient in need thereof by applying the microneedle array to a skin of the patient, and pressing to effect the penetration of the microneedles into the skin. The array may then be bandaged to retain in place, or the backing layer may be washed off by gentle rubbing of the array with soap and warm water, to decrease the sensation of the foreign object. If needed, the array may be removed, e.g., to stop the drug action, or to avoid development of adverse reactions.

Usually, the array may be applied as needed, e.g., up to several times a day. However, it may be advantageous to utilize microneedle arrays that contain a significant absolute drug loading and release the drug over an extended time interval. Therefore, the array may be applied at any suitable frequency as known in the art, e.g., three times a day, twice a day, once daily, and also once every two days, twice a week, or even once a week.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the scope of the appended claims. The herein described preferred embodiments and the drawings provided herein demonstrating some of the embodiments of the present invention are provided to better understand the present invention, which however does not limit the invention in any respect.

Several further points must also be noted in reference to this specification and the appended claims. All scientific and technical terms used herein have meanings commonly used in the art unless otherwise specified. The definitions as provided herein are given with the purpose to facilitate understanding of certain terms used frequently herein and are not necessarily meant to limit the scope of the present disclosure. As used herein the term “about”, “c.a.”, “˜”, and like, as used interchangeably herein, refers to the value and the range of ±10%. The terms “comprises”, “comprising”, “includes”, “including”, “having” and their conjugates mean “including but not limited to”. This terms also encompass the terms “consisting of” and “consisting essentially of”, which have their narrower meaning as known in the art. It must also be noted that the singular forms “a”, “an”, and “the”, include plural referents, unless the content clearly dictates otherwise. As used herein, a phrase in the form “A and/or B” means a selection from the group consisting of (A), (B) or (A and B). As used herein, a phrase in the form “at least one of A, B, and C” means a selection from the group consisting of (A), (B), (C), (A and B), (A and C), (B and C) or (A, and B, and C).

It is appreciated that certain features of the invention, which are, for brevity, described in the context of separate embodiments, may also be provided in combination with other described features in a single embodiment. Conversely, certain features described as specific combinations of various sub-features, which are, for clarity and demonstration, are described in the context of a single embodiment, may also be provided as separate embodiments individually or in any suitable sub-combination with other features and/or embodiments as suitable and operative. Certain features described in the context of various embodiments are not to be considered essential features of those embodiments, unless the embodiment is inoperative without those elements.

### EXAMPLES

### Materials

Furosemide (>98%) was purchased from Apollo Scientific Ltd (UK). PLGA (Purasorb PDLG 5010, 50:50, MW 100,000) was donated by Corbion Purac (Gorinchem, the Netherlands). Silicone MPatch microneedle templates were purchased from Micropoint Technologies Pte Ltd. (Pioneer Junction, Singapore) and were pyramidal in shape with a dimension of 10×10 needle array, 200 μm base, 500 μm height and 500 μm pitch (the distance between the microneedles' apexes). Phosphate buffer saline (PBS) was purchased from Hyclone Laboratories. Organic solvents were obtained from Sigma-Aldrich (Rehovot, Israel). Sodium alginate, manufactured by Fischer Chemical, was supplied by Holland Moran, Israel.

### Example 1—Preparation of Microneedle Array

Furosemide-loaded PLGA MNs were prepared by a two-step mold casting technique. First, a common solution in chloroform of PLGA and furosemide, in varying ratios, was cast onto the mold to fill the needles, and then an aqueous solution of sodium alginate with glycerin was added and dried in a desiccator. Different microneedles (MNs) formulations were prepared as shown in Table 1.

Briefly, a 5% w/v of PLGA in chloroform containing varying concentrations of furosemide were left stirring until a clear solution was obtained. Then a 150 μL aliquot of the solution was cast onto the polydimethylsiloxane (PDMS) microneedle molds and degassed in vacuo for 2 min, to remove air and thus fill the mold cavities, simultaneously partially evaporating the solvent. The back-layer solution was prepared, comprising of 2% w/v sodium alginate and 1% w/v glycerol in distilled water (DW). An aliquot of 100 μL of the backing layer solution was added to the molds, effecting the phase-transition of PLGA and forming of the needles. The templates were kept overnight in a desiccator. Lastly, the MNs patches were peeled off from the mold and were stored in a desiccator until future use. The blank PLGA MNs (P-MN) were prepared by the same procedure with FUR-loaded MNs without adding furosemide at any stage of the preparation.

TABLE 1
Composition of PLGA MNs.

| | | | |
|---|---|---|---|
| P-MN | FUR-MN2 | FUR-MN3 | FUR-MN4 |

Microneedles

| | | | | | | | | |
|---|---|---|---|---|---|---|---|---|
| Furosemide | | | 3.75 | mg | 2.50 | mg | 1.87 | mg |
| PLGA | 7.5 | mg | 7.5 | mg | 7.5 | mg | 7.5 | mg |

Backing layer

| | | | | | | | | |
|---|---|---|---|---|---|---|---|---|
| Sodium | 2.0 | mg | 2.0 | mg | 2.0 | mg | 2.0 | mg |
| alginate | | | | | | | | |
| Glycerin | 1.0 | mg | 1.0 | mg | 1.0 | mg | 1.0 | mg |

Volatiles

| | | | | | | | | |
|---|---|---|---|---|---|---|---|---|
| Chloroform | 150 | μL | 150 | μL | 150 | μL | 150 | μL |
| DDW | 100 | μL | 100 | μL | 100 | μL | 100 | μL |

Formulation characteristics

| | | | | |
|---|---|---|---|---|
| FUR:PLGA | 0 | 1:2 | 1:3 | 1:4 |
| C_{PLGA} | 7.5% | 7.5% | 7.5% | 7.5% |
| C_{alginate} | 2.0% | 2.0% | 2.0% | 2.0% |

### Example 2—Mechanical Characterization of the Microneedle Array

The microneedle array obtained in the example 1 was further characterized.

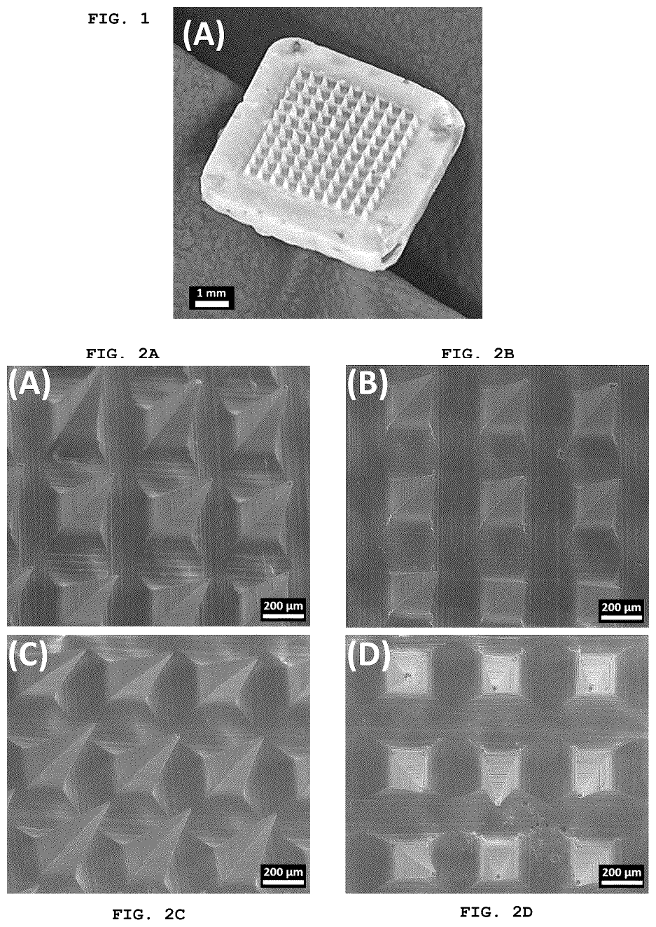

Morphological characterization of FUR-loaded MNs was carried out by visual inspection and using an Environmental Scanning Electron Microscope (ESEM) (Quanta 200, FEI, Germany) to evaluate the MNs surface morphology and dimensions. Upon visual inspection, MNs were observed as a 10×10 array and the shape of needle tips used in manufacture of polymer MNs was quadrangular pyramid (FIG. 1). In the figure, a photograph is presented wherein a plurality of protruding pyramidal needles are observed, with the lighting applied from upper-left corner of the photograph. The size bar at lower left corner indicates the size of 1 millimeter, as designated by the label “1 mm”. In the FIG. 1, MN array demonstrating to main parts could be seen: the furosemide-loaded biodegradable needles made of PLGA, and the flexible back layer containing sodium alginate and glycerol.

When characterized by SEM, all MN formulations had a quadrangular pyramidal shape and were uniformly distributed on the substrate (FIGS. 2A to 2D). The size bar at lower right corner indicates the size of 200 micrometer, as designated by the label “200 μm”. P-MN, FUR-MN2, FUR-MN3 and FUR-MN4 are depicted at the FIGS. 2A, 2B, 2C, and 2D, respectively, with the micrographs A-C demonstrating the microneedles view from lower left towards upper right of the micrograph (c.a. from about 60° above and about 45° angle relative to the microneedle base), and micrograph D represents microneedles from upper part downwards of the micrograph, i.e., directly from above. The MNs were successfully prepared showing dimensions of 494.35-499.21 μm height and 196.62-199.31 μm width of the base as depicted in Table 2. Additionally, the SEM images confirmed intact sharp tips of the MNs without visible cracks, which is needed for the proper piercing of the skin.

TABLE 2
the dimensions of individual microneedles

| Formulation | Base (μm) | Height (μm) |
|---|---|---|
| P-MN | 198.76 ± 3.81 | 494.35 ± 13.46 |
| FUR-MN2 | 199.31 ± 3.50 | 499.21 ± 13.78 |
| FUR-MN3 | 197.65 ± 3.18 | 496.78 ± 10.92 |
| FUR-MN4 | 196.62 ± 3.88 | 497.59 ± 11.60 |

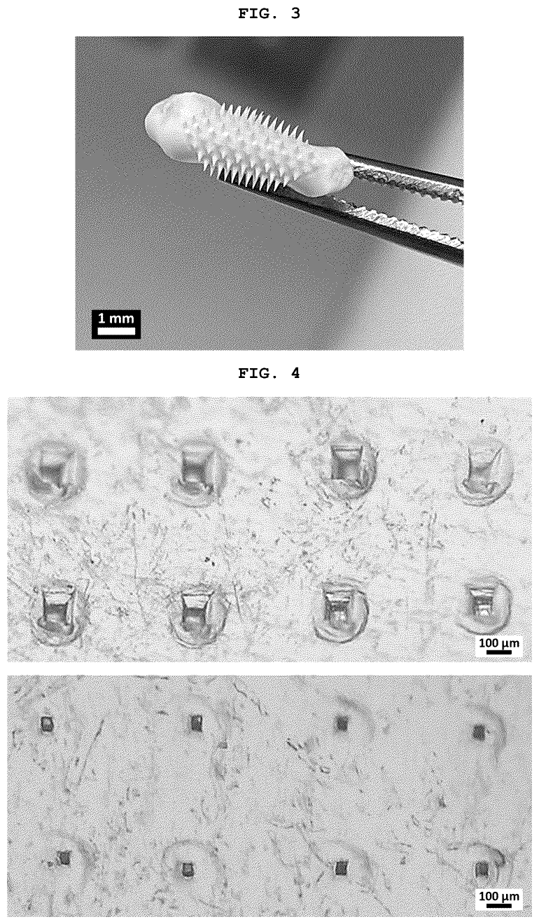

The flexibility of the different PLGA MNs was assessed manually by folding each patch (i.e., microneedle array) repeatedly at the same place using the thumb and forefinger until the patch broke or visible cracks appeared. Three patches of each type were taken for the test. The folding endurance test was carried out to evaluate the strength of the patches, wherein the MNs were successfully folded more than 300 folds without presenting any damage or cracks, suggesting that all developed MNs were flexible and exhibited adequate mechanical properties. The obtained MNs patch was flexible that can be easily folded without damaging MN arrays (FIG. 3). The size bar at lower left corner indicates the size of 1 millimeter, as designated by the label “1 mm”. Without being bound by a particular theory it is currently believed that this high flexibility is due to the use of glycerol as a plasticizer in the back layer, whereas the alginate-PLGA blend on the interphase is responsible for the superior resilience.

The uniformity of mechanical strength of MNs on array is an essential indicator of uniform channels created in skin. The piercing ability and uniformity was assessed using a stack of ten layers of Parafilm™ laboratory sealant. In this study, ten layers of Parafilm™ (thickness of 140±10 μm) were stacked together as a skin simulant for MN insertion to evaluate the mechanical uniformity of the obtained MNs. The microneedle arrays were applied onto the stack using a thumb, for 30 seconds, and the individual layers were thereafter observed under an optical microscope to study the mechanical properties of the needles. The mechanical uniformity of PLGA MNs was estimated from the dimensions of 10 randomly selected pores created in the first (140 μm) layer and the second (280 μm) layer, using ImageJ software (National Institute of Health, Bethesda, MD, USA). It could be readily observed that MNs created 100 square-shaped pores in the first and the second Parafilm™ layer, whereas the dimensions of the square pores were lower than the needle base (Table 3, n=10, mean±s.d.), probably due to a certain degree of elasticity of the substrate. As seen in the table 3 and FIG. 4, the size of the pores decreased from the first to second Parafilm™ layer, following the pyramidal shape of the MNs. Furthermore, the minimal standard deviation of these demonstrated the uniformity of created pores as well as the mechanical uniformity of the needles. Moreover, the size of the created pores roughly coincides with the dimensions of the axial cross-section of the microneedles at the selected thickness. In FIG. 4, micrographs of series of perforations are demonstrated, corresponding to the first and the second layer of Parafilm™, at the top and bottom of the FIG. 4. The size bar at lower right corner indicates the size of 100 micrometer, as designated by the label “100 μm”.

TABLE 3

| Formulation | 1st Layer Length (μm) | 2nd Layer Length (μm) |
|---|---|---|
| FUR-MN2 | 78.95 ± 2.25 | 39.17 ± 4.47 |
| FUR-MN3 | 79.21 ± 4.57 | 39.28 ± 4.37 |
| FUR-MN4 | 79.52 ± 4.91 | 45.86 ± 4.33 |

Close to 100% of the microneedles in the array were able to pierce the first layer, and between 70 and 80 percent were also able to penetrate the second layer as well. The height of the microneedles was measured using SEM after the piercing test, and the value diminished slightly, between 7 and 10%, with the greatest height reduction observed with MN4, having the lowest drug loading.

These results of successful insertion of MNs into Parafilm™ layers suggested that MNs would easily penetrate consistently and reproducibly following thumb pressing across the outermost layer of the skin, the stratum corneum, which is usually about 50 μm thick in a healthy human. This would permit intradermal insertion of the MNs, allowing drug delivery across the skin.

The microneedles were forced into the chicken skin and the displacement force was recorded. Similar test was carried out on a solid metal block. The results indicated that the needles became compressed only after they were displaced to the values corresponding the penetration through the skin, corroborating the skin penetration of the microneedles indeed occurs, as predicted by the Parafilm™ tests.

### Example 3—Surface pH of the Microneedles

A highly acidic or alkaline pH can cause skin irritation, therefore, the surface pH of the prepared MNs was determined. The surface pH of three prepared microneedle arrays of each formulation was determined after placing in glass tubes containing 10 ml of double distilled water for 2 h at room temperature. A combined glass electrode was located near the surface of the film to be measured and pH measurements were performed after equilibration time of 1 min. The surface pH of all the samples evaluated was in the range of 6.03±0.01 to 6.24±0.09 (Table 4), indicating that no skin irritation is expected upon their application.

TABLE 4

| Formulation | Surface pH |
|---|---|
| FUR-MN2 | 6.03 ± 0.01 |
| FUR-MN3 | 6.09 ± 0.01 |
| FUR-MN4 | 6.24 ± 0.09 |

### Example 4—Drug Properties in the Microneedles

The drug loading has been determined to evaluate the encapsulation efficiency of the process of microneedles' production. The furosemide-loaded MNs were dissolved in 1 mL acetone. Methanol was then added to precipitate the polymers, followed by vortex and centrifugation for 2 min at 2000 rpm. Afterwards, the supernatant containing the drug and organic solvents was transferred and evaporated. The residue was then dissolved in methanol and furosemide was quantitatively determined, after suitable dilutions with the same solvent, using a Biochrom UV-vis spectrophotometer at a wavelength of 275 nm. The calibration curve was linear in the concentrations of between 0 and 20 μg/mL (R²=0.997). High encapsulation efficiency was obtained for FUR-MN formulations, with an observed decrease as the drug: polymer ratio decreased. Still, drug loading content ranged from 19.1±1% for FUR-MN4 to 28.9±1.4% for FUR-MN2 (Table 5, n=3, mean±s.d.). The encapsulation efficiency in percentage (%) was determined by dividing the amount of drug recovered from MN, by the amount of drug supplied initially, and multiplying by 100. Drug loading content percentage (%) was determined as the amount of drug determined in the microneedles divided by the total amount of drug and the polymer initially supplied, multiplied by 100.

TABLE 5

| | Encapsulation | Drug loading |
|---|---|---|
| Formulation | efficiency (%) | content (%) |
| FUR-MN2 | 81.5 ± 5.8 | 28.9 ± 1.4 |
| FUR-MN3 | 91.5 ± 4.6 | 23.4 ± 0.9 |
| FUR-MN4 | 94.2 ± 6.1 | 19.1 ± 1.0 |

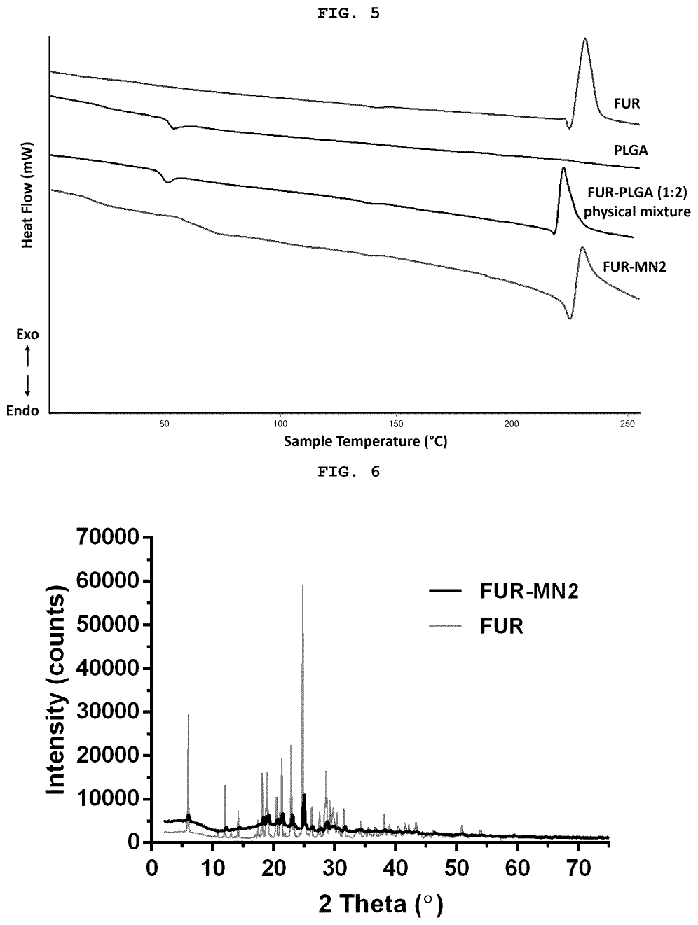

Differential scanning calorimetry measurements were performed to determine the solid-state form of the drug inside the microneedles. The DSC measurements were performed using a DSC 1 Star System equipped with Star Software (Mettler Toledo, Greifensee, Switzerland) and a DSC131 Evo (SETARAM Instrumentation, Caluire-etCuire, France). Accurately weighed samples of 5-12 mg were placed in aluminum pans, and then the samples were scanned from −20° C. to 270° C. at a constant heating rate of 10° C./min under continuous dry nitrogen flow. The analysis was performed for PLGA, furosemide, furosemide-PLGA (1:2) physical mixture, and FUR-MN2. The thermogram is presented in FIG. 5. In the FIG. 5, heat flux is charted along the ordinate axis, labelled as “Heat Flow (mW)”, versus the temperature charted along the abscissa axis, labelled as “Sample Temperature (° C.)”. The upwards and downwards arrows at the ordinate axis denote exotherms and endotherms, respectively, as denoted by the “Exo↑”, and “Endo↓”, respectively. Furosemide thermogram is presented first from top, denoted as “FUR”, followed by PLGA (denoted as “PLGA”), physical mixture of furosemide and PLGA 1:2 (denoted as “FUR-PLGA 1:2 physical mixture”), and the formulation FUR-MN2 (denoted as “FUR-MN2”).

The DSC thermogram of PLGA shows a glass transition temperature (Tg) at 49.5° C. The thermogram of furosemide exhibited a characteristic melting endotherm at 221.2° C., followed by an exothermic peak at 224.7° C., which indicates the crystalline nature of the drug. The peak corresponding to the melting point of furosemide in FUR-MN2 is less defined and broader indicating a decline in the crystallinity of FUR in the MNs. This could be ascribed to a partial drug amorphization occurred during solidification within PLGA matrix, which is supported by the disappearance of the defined glass transition point of the PLGA, and emergence of two further thermal transitions, one of which might be attributed to alginate.

To corroborate the finding that furosemide was present in at least partially crystalline form inside the microneedles, powder X-ray diffraction (PXRD) measurements were performed, using the D8 Advance diffractometer (Bruker AXS, Karlsruhe, Germany) with secondary Graphite monochromator, 2° Sollers slits and 0.2 mm receiving slit. Low-background quartz sample holders were carefully filled with the powdered samples. XRD patterns within the range 2° to 75° 2θ were recorded at room temperature using CuKα radiation (λ=1.5418 Å) with following measurement conditions: tube voltage of 40 kV, tube current of 40 mA, step-scan mode with a step size of 0.02° 20 and counting time of 1 s/step. The PXRD patterns of furosemide alone and loaded into PLGA MNs are depicted in FIG. 6. In the FIG. 6, the number of counts, expressed as intensity of the radiation, is charted along the ordinate axis, labelled as “Intensity (Counts)”, versus the 2θ angle along the abscissa axis, labelled as “2 Theta) (°)”. Furosemide diffractogram is presented first from bottom, denoted as “FUR” in the legend and lighter line, followed upwards by the formulation FUR-MN2 (denoted as “FUR-MN2”) and the darker line.

The presence of numerous distinct peaks in both samples indicates that FUR was present in its crystalline form with major characteristic diffraction peaks appearing at a diffraction angle of 2θ (i.e., at 6.02, 12.05, 18.12, 18.96, 20.49, 21.35, 22.91 and 24.79). Nevertheless, in the case of the FUR-MN2, the intensity of the peaks was reduced. This, together with the less intense melting peak observed in the DSC experiment, indicate a degree of amorphization of the drug within the PLGA matrix.

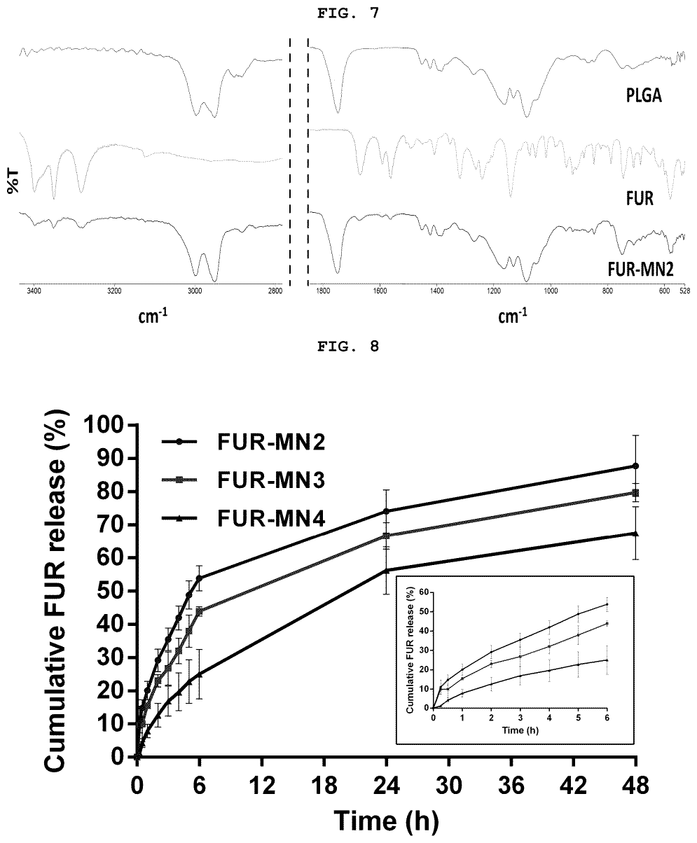

Attenuated total reflectance Fourier-transformed infra-red (ATR-FTIR) spectra were obtained by a PerkinElmer Spectrum 100S spectrometer equipped with a universal ATR sampling accessory. The transmission spectra were recorded over the range between 500-4000 cm⁻¹, using 4 scans with a resolution of 4 cm⁻¹. PLGA, furosemide, and FUR-MN2 were measured (FIG. 7). In the FIG. 7, the amount of transmitted infrared radiation is charted along the ordinate axis, labelled as “% T”, versus the wavelength expressed in reciprocal centimeter, labelled as “cm⁻¹”, along the abscissa axis. PLGA spectrum is presented first from top, denoted as “PLGA”, followed downwards by furosemide, denoted as “FUR”, followed by the formulation FUR-MN2 (denoted as “FUR-MN2”). Shaded area contains the characteristic peaks as described below.

Characteristic peaks of furosemide at 3400 cm⁻¹, 3260 cm⁻¹, 1665 cm⁻¹, and 1560 cm⁻¹ were distinctly observed. The 3400 cm⁻¹ band shows the NH₂ stretching vibration of the aromatic ring and the 3260 cm⁻¹ band shows the SO₂NH₂ stretching vibration, while the 1665 cm⁻¹ band shows the bending vibration of the amine group. The 1560 cm⁻¹ band is assigned to the asymmetric stretching vibration of the carbonyl group. The characteristic peak of PLGA at 1750 cm⁻¹ due to the ester group was also distinctly observed. No significant difference shift in the position of the absorption peaks was observed for the microneedles. The observation of the characteristic peaks of furosemide in MNs indicates that it does not interact with other excipients used in the formulations, including the matrix-forming PLGA.

### Example 5—Dissolution Studies

To evaluate the controlled-release potential of the microneedles' formulations and to demonstrate the release of the drug therefrom, dissolution studies have been performed. Samples of FUR-MN2, FUR-MN3 and FUR-MN4 were placed into 10 mL of PBS (pH 7.4) and maintained at 37° C. in a rotary shaker incubator (50 rpm). At predetermined time intervals, 1 mL samples were withdrawn from the release medium and were replenished immediately with the same volume of fresh pre-warmed PBS (37° C.), maintaining sink condition throughout the experiment. Furosemide concentrations were determined as in Example 4 above, with the calibration curve in PBS (pH 7.4) being linear in the range from 0 to 30 μg/mL (R²=0.996).

The percentage of furosemide release over time is demonstrated in the FIG. 8 (as mean±s.d. of four experiments). In the FIG. 8, the cumulative release of furosemide, expressed as percentile of the drug loading, is charted along the ordinate axis, labelled as “Cumulative FUR Release (%)”, versus the time elapsed from the beginning of the experiment expressed in hours, along the abscissa axis, labelled as “Time (h)”. Release profile of the formulations FUR-MN2 (denoted as “FUR-MN2” in the legend and the closed circles (●) on the graph), FUR-MN3 (denoted as “FUR-MN3” in the legend and the closed squares (▪) on the graph), and FUR-MN4 (denoted as “FUR-MN4” in the legend and the closed triangle (▴) on the graph), are shown. The inset figure describes in similar way the initial release profiles between 0 and 6 hours.

The data obtained from in vitro release studies was fit by several kinetic models, to determine the mechanism of drug release from the films. Correlation coefficient (R²) values of various release kinetic models for microneedles containing varying concentrations of furosemide is summarized in Table 6. The release kinetics of furosemide from the film showed the best fit for first order kinetics and the release exponents obtained from Korsmeyer-Peppas model were 0.45<n<0.89, suggesting that for the higher-loading the drug release followed Fickian diffusion model, whereas for the lower-loading formulation an anomalous non-Fickian transport was observed.

TABLE 6

| | Zero order | First order | Korsmeyer-Peppas model | |
|---|---|---|---|---|
| Formulation | R² | R² | n | R² |
| FUR-MN2 | 0.717 | 0.930 | 0.502 | 0.996 |
| FUR-MN3 | 0.788 | 0.934 | 0.496 | 0.969 |
| FUR-MN4 | 0.877 | 0.949 | 0.801 | 0.946 |

Furosemide release pattern from MNs exhibited a small initial “burst” release for formulations with higher loading, accompanied by a more sustained release phase, and the cumulative drug release over 48 hours increased by incorporating higher initial drug loading.

1. A flexible microneedle array comprising a plurality of microneedles and a backing layer,
wherein said microneedles comprise a polyester selected from the group consisting of polylactic acid, polyglycolic acid, poly(lactic-co-glycolic) acid, polycaprolactone, and combinations of any of these,
wherein said microneedles further comprise a pharmaceutically active agent that is soluble in a solvent capable of dissolving said polyester, and that is poorly soluble in distilled water at 25° C., and
wherein said backing layer comprises an alginate and a polyol.

2. The flexible microneedle array according to claim 1, wherein said pharmaceutically active agent is essentially homogenously distributed within said microneedles, such that the concentration of said pharmaceutically active agent at the distal ⅓ portion of said microneedles is between 50% and 150% of that in the proximal ⅓ portion of said microneedles, relative to the base of said microneedles and said backing.

3. The flexible microneedle array according to claim 1, wherein said pharmaceutically active agent and said polyester are individually or concomitantly essentially homogenously distributed within said microneedles, such that the concentration of said pharmaceutically active agent and/or said polyester at the distal ⅓ portion of said microneedles is between 50% and 150% of that in the proximal ⅓ portion of said microneedles, relative to the base of said microneedles and said backing layer.

4. The flexible microneedle array according to claim 1, wherein said backing layer is flexible such that no cracks are formed when said microneedle array is bent over a central axis of said microneedle array by repetitively bringing together the opposing edges of the backing layer over 50 times.

5. The flexible microneedle array according to claim 1, wherein said solvent capable of dissolving said polyester comprises chloroform, dichloromethane, or acetone.

6. The flexible microneedle array according to claim 1, wherein a weight ratio between said polymer and said pharmaceutically active agent is between 1.5:1 and 5:1.

7. The flexible microneedle array according to claim 1, wherein a weight ratio between said polymer and said pharmaceutically active agent is between 2:1 and 4:1.

8. The flexible microneedle array according to claim 1, wherein pharmaceutically active agent is selected from the group consisting of furosemide, levosimendan, dobutamine, amiodarone, metoprolol, lidocaine, and chlorhexidine.

9. The flexible microneedle array according to claim 1, wherein pharmaceutically active agent is furosemide.

10. The flexible microneedle array according to claim 1, wherein a weight ratio between said alginate and said polyol in said backing layer is between 1:1 and 3:1.

11. The flexible microneedle array according to claim 1, wherein a weight ratio between said alginate and said polyol in said backing layer is between 1.8:1 and 2.3:1.

12. The flexible microneedle array according to claim 1, wherein said polyol is selected from the group consisting of glycerin, sorbitol, xylitol, and combinations of any of the above.

13. The flexible microneedle array according to claim 1, wherein said polyol is glycerin.

14. The flexible microneedle array according to claim 1, wherein said polyester is poly-lactic-co-glycolic acid, wherein said pharmaceutically active agent is furosemide, and wherein said polyol is glycerin.

15. The flexible microneedle array according to claim 14, wherein a weight ratio between said poly-lactic-co-glycolic acid and said furosemide is between 2:1 and 4:1.

16. The flexible microneedle array according to claim 14, wherein a weight ratio between said alginate and said glycerin in said backing layer is between 1.8:1 and 2.3:1.

17. A method of manufacturing a microneedle array, comprising combining on a microneedle array mold an organic solution of a polymer forming the microneedles and an aqueous solution of a polymer forming a backing layer of said microneedle array.

18. The method according to claim 17, comprising
providing a mold for microneedle array,
providing a microneedle precursor solution comprising a mutual solution of a polymer and a pharmaceutically active agent in a solvent,
applying said microneedle precursor solution to said mold,
optionally degassing and/or partially evaporating said microneedle precursor solution on said microneedle mold, and
applying an aqueous backing layer solution,
wherein said aqueous backing layer solution comprises an alginate and a polyol, and
wherein said applying of said aqueous backing layer solution effects forming microneedles from said microneedle precursor solution in said mold, said microneedles comprising said polymer and said pharmaceutically active agent.

19. The method according to claim 18, further comprising drying said mold after said applying of said aqueous backing layer solution.

20. The method according to claim 17, further comprising sterilizing said microneedle array.

21. The method according to claim 17, further comprising packing said microneedle array in a suitable package.

22. The flexible A microneedle array according to claim 1, for use in treatment of a heart disease or a vascular disease in a patient in need thereof.

23. The flexible microneedle array according to the claim 22, wherein said microneedle array is applied to the skin of said patient.

24. The flexible microneedle array according to claim 22, wherein said microneedle array is applied to the patient in need thereof in a frequency selected from the group of thrice daily, twice daily, once daily, thrice weekly, twice weekly, and once weekly.
